(19) United States
(12) Patent Application Publication
(10) Pub. No.: US 2025/0145954 A1
(43) Pub. Date: May 8, 2025

(54) EXPANSION OF HUMAN HEMATOPOIETIC STEM CELLS FROM HUMAN YELLOW BONE MARROW

(71) Applicant: UNIVERSITY OF MASSACHUSETTS, Boston, MA (US)

(72) Inventors: Tammy NGUYEN, Worcester, MA (US); Silvia CORVERA, Shrewsbury, MA (US); Tiffany DESOUZA, Putnam, CT (US); Anand DESAI, Natick, MA (US)

(21) Appl. No.: 18/833,774

(22) PCT Filed: Jan. 30, 2023
(86) PCT No.: PCT/US23/11854

§ 371 Date: Jul. 26, 2024

Related U.S. Application Data
(60) Provisional application No. 63/304,247, filed on Jan. 28, 2022.

(51) Int. Cl. C12N 5/0789; A61K 35/17; C12N 5/078

(52) U.S. Cl. CPC C12N 5/0647; A61K 35/17; C12N 5/0634; C12N 2501/105; C12N 2501/11; C12N 2501/115; C12N 2501/125; C12N 2501/165; C12N 2501/2306; C12N 2501/26; C12N 2501/999; C12N 2533/50

(57) ABSTRACT

Hematopoietic stem cells (HSC) are essential to developing a functional immune system, including monocyte-macrophage cells required for wound healing. Described herein are methods of using yellow bone marrow as a source of cells to generate hematopoietic stem cells. The methods include expansion of human hematopoietic stem cells by culture of human yellow bone marrow in 3-dimensional hydrogel and pro-angiogenic, stem cell-promoting medium.

### CLAIM OF PRIORITY

This application claims the benefit of U.S. Provisional Patent Application Ser. No. 63/304,247, filed on Jan. 28, 2022. The entire contents of the foregoing are hereby incorporated by reference.

### TECHNICAL FIELD

Described herein are methods of using yellow bone marrow as a source of cells to generate hematopoietic stem cells. The methods include expansion of human hematopoietic stem cells by culture of human yellow bone marrow in 3-dimensional hydrogel and pro-angiogenic stem cell-promoting medium.

### BACKGROUND

Hematopoietic stem cells (HSC) are essential to developing a functional immune system, including monocyte-macrophage cells required for wound healing.

### SUMMARY

Provided herein are methods of obtaining a population of hematopoietic stem cells (HSCs) from a subject. The methods comprise providing a sample comprising yellow bone marrow from a subject; dividing the sample into pieces, e.g., each piece about 0.5-2 mm³, preferably about 1 mm³; embedding the pieces in a hydrogel with media (i.e., a 3-dimensional culture) for expansion of HSCs; maintaining the pieces in culture for a time sufficient for expansion of the HSCs, and isolating the HSCs from the hydrogel, thereby obtaining a population of HSCs. Red bone marrow can alternatively be used in the methods and compositions described herein.

In some embodiments, the sample is from a long bone of the subject. In some embodiments, the subject is a mammal, e.g., a human.

In some embodiments, the hydrogel comprises MATRIGEL.

In some embodiments, the media for expansion of HSCs comprises pro-angiogenic factors, preferably EGF, IGF, VEGF, and FGFb, and stem cell promoting factors, preferably stem cell factor (SCF), and optionally one or more of thrombopoietin (TPO), fms related receptor tyrosine kinase 3 (Flt3), Interleukin 6 (IL6), and an aryl hydrocarbon receptor (AHR) antagonist, and UM729. In some embodiments, the AHR antagonist is StemRegenin 1 (SR1).

In some embodiments, the pieces are maintained in culture for at least 10, 12, or 14 days, or up to 17, 18, 20, or 24 days.

In some embodiments, the methods include isolating cells that are CD34+, CD45dim, CD90+, CD38−, and/or Lineage−, optionally using cell sorting or magnetic beads.

In some embodiments, the population comprises 10,000-20,000 HSCs.

In some embodiments, the methods further include culturing the population of HSCs under conditions sufficient for differentiation into erythroid progenitor cells, granulocyte-macrophage progenitor cells, multipotential granulocyte, erythroid, macrophage, megakaryocyte progenitor cells, and/or lymphoid progenitor cells (Common Lymphoid Progenitors (CLPs)), or to B cells, T cells, or NK cells.

Also provided herein are methods of treating a subject, the method comprising: obtaining a population of HSCs by a method described herein; optionally differentiating the HSCs into a selected erythroid, lymphoid, or myeloid cell type; and administering the differentiated cells to the subject. In some embodiments, the yellow bone marrow is obtained from the subject to be treated.

In some embodiments, the subject is diabetic and has a wound, and the cells are differentiated, e.g., to immune cells, preferably to monocytes and/or macrophages, and administered to the wound.

Additionally provided herein are populations of HSCs obtained by a method described herein.

The population of HSCs of claim 14, for use in a method of treating a subject.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a schematic of exemplary methods for human bone marrow tissue explant collection and in vitro cell expansion.

FIG. 2 is a set of images of tissue explants from lower limb subcutaneous fat tissue explant (BMS), bone marrow red (BMR), and bone marrow yellow (BMY) in the three-dimensional culture system described herein at 3 days and 14 days in culture under 5× magnification. De novo sprouting cell expanded from 1 mm³ tissue explant was observed on Day 14. Scale bar is 10 um.

FIGS. 3A-B. 3A, a graph showing distribution of numbers of cells recovered from the three-dimensional culture system after 14-17 days in culture for lower limb subcutaneous fat tissue explant (BMS), bone marrow red (BMR), and bone marrow yellow (BMY) from respective donors. 3B, a graph showing average number of tissue explant expanded cells collected after 14-17 days for growth. Tissue explant cell expansion was extracted from Matrigel using a digestion process to generate a single cell suspension. Manual quantification of cell number recovered from Matrigel digestion process was done using a hemocytometer and microscopy imaging at 10× objective.

FIG. 4 is a set of graphs of flow cytometry experiments for identification and quantification of hematopoietic stem cells in human bone marrow tissue explant cell culture. Tissue explant cell expansion was extracted from Matrigel using a digestion process to generate a single cell suspension. Single cell suspension from BMR, BMY, and BMS tissue explant cell culture is incubated with fluorescent antibodies that bind to common cell surface receptor markers used to identify hematopoietic stem cells (HSC defined as CD34+, CD38−, CD90+, and low levels of CD45+). Cells with fluorescence pattern for HSC are outlined in black boxes with indicated cell percentage of HSC in total cell population.

FIGS. 5A-B. 5A, a graph showing percentage of CD34+ cells obtained from the three-dimensional culture system after 14-17 days in culture for lower limb subcutaneous fat tissue explant (BMS), bone marrow red (BMR), and bone marrow yellow (BMY) from respective donors. 5B, a graph showing average percent of CD34+ cells collected after 14-17 days for growth.

FIG. 6 is a set of images of erythroid and myeloid colonies formed from CD34+ cells isolated from human bone marrow tissue explant cultures. Subcutaneous fat (BMS), yellow bone marrow (BMY), and red bone marrow (BMR) tissue explants cells harboring CD34 receptors (conical marker for hematopoietic stem cells, HSC) were isolated using magnet beads (StemCell, Cat #17856). Examples of erythroid cell colonies are indicated by black arrows and myeloid colonies are indicated by white arrows. Scale bar is 10 um.

FIG. 7 shows quantification of erythroid and granulocyte-myeloid-macrophage colonies from CD34+ Cells isolated from human bone marrow tissue explant culture from subcutaneous fat (BMS), yellow bone marrow (BMY), and red bone marrow (BMR) tissue explants. Examples of erythroid cell colony (CFU-E) and granulocyte-myeloid-macrophage (CFU-GEMM) forming colony are shown to the right. N=individual 3 donors.

FIG. 8 shows results of principal component analysis (PCA) of differential gene expression between red and yellow bone marrow. N=individual 4 donors.

FIG. 9 is a plot showing selected differentially expressed genes between red and yellow bone marrow. Bulk RNA-Seq was performed on cells expanded from yellow bone marrow (BMY) and red bone marrow (BMR) tissue explants after 14-17 days in culture from 4 patient donors. Genes that are differentially expressed higher in BMR are indicated in red and genes that are differentially expressed higher in BMY are indicated in orange. N=individual 4 donors.

FIG. 10 is a graph showing leptin receptor gene expression levels determined in cells generated from red and yellow bone marrow. Expression fold change was normalized to housekeeping gene RPL4 and relative to the lowest expressing sample. Data represents technical duplicates from 3 different human donors.

FIG. 11 is a set of fluorescent activated cell sort (FACS) graphs showing the results of in vitro differentiation of human bone marrow-derived HSCs into monocytes and macrophages. Representative fluorescent activated cell sort (FACS) analysis of in vitro cytokine induction of HSCs (CD34⁺ CD38⁻) cells derived from BMY and BMR tissue explants and human umbilical cord blood (HUCB). HSC cell population percentage represents CD34⁺CD38⁻ cells in oval. Monocyte cell population percentage represents CD45⁺CD14⁺ cells in oval. M1 Macrophage cell population percentage represents CD45⁺CD64⁺ in oval. M2 Macrophage cell population percentage represents CD45⁺CD163⁺ cells in oval.

### DETAILED DESCRIPTION

Hematopoietic stem cells (HSC) are a self-renewing, rare population of cells that give rise to all cellular components of the blood, including red blood cells and immune cells. Human HSCs are used to repair the immune system; for example, a current treatment of leukemia consists in ablating the bone marrow; and then reconstituting it with compatible HSCs from donors who are free of cancer. HSCs can also be used to repair genetic immunodeficiencies or disorders of hemoglobin function. More recently, gene-corrected HSCs, generated using CRISPR-Cas9 methods, are arising as promising therapeutics for a large number of disorders.

HSCs are currently obtained from the patient's blood after mobilizing the immune system by injection of growth factors such as G-CSF. This process incurs risks associated with 4 to 5 days of subcutaneous injections of human granulocyte colony-stimulating factors (G-CSFs), followed by up to 2 to 5 additional days for peripheral blood dialysis to concentrate stem cells. The risk of G-CSFs includes serious allergic reactions, respiratory distress, splenic rupture, sickle cell disorders, kidney injury, inflammation, and potential increase in malignancy. HSCs can also be collected by surgical biopsy from the red bone marrow of the iliac crest, but this process required anesthesia and yielded very few cells, and therefore has been largely supplanted by G-CSF mobilization.

Described herein are methods to obtain and expand human HSCs from small samples of human yellow bone marrow. The yellow bone marrow is localized in the shaft of long bones and contains adipose tissue. Previously, it was thought that populations of HSCs in sufficient amounts were restricted to the adipose-poor red bone marrow; such as that in the iliac crest. The present disclosure shows that HSCs are present in the yellow marrow in numbers comparable to (or even higher than) red marrow; and can be expanded in vitro.

Because yellow marrow is much more abundant that red marrow in adults, and can be accessed easily through core biopsies, the present methods can be used to recover many more HSCs than previously possible. In addition, the present methods allow for expanded HSCs to be banked for future use, therefore only requiring a one-time sample collection.

The bone marrow culture expansion methods described herein expand human hematopoietic stem cells from bone marrow compartments (red and yellow marrow) using 3-dimensional culture in a matrix in a media formulation that contains specific growth factors and micronutrients. Briefly, the process uses human bone marrow harvested from yellow marrow that can be obtained through a core needle bone biopsy or surgically removed human bone. In preferred embodiments, the present methods do not include injection of growth factors such as G-CSF.

Preferably, the bone marrow samples are cut into pieces, each about 0.5-2 mm³, or about 1 mm³, and embedded in a tissue culture dish coated with a 3-dimensional hydrogel and media for expansion. In some embodiments, the samples are 1-20 g, preferably 2-15, or 4-14 g.

The hydrogel comprises a natural or synthetic hydrogel scaffold, e.g., comprising natural extracellular matrix (ECM), e.g., MATRIGEL (Corning, Corning, NY), GELTREX LDEV-Free Reduced Growth Factor Basement Membrane Matrix (GIBCO/ThermoFisher), or CULTREX Basement Membrane Extract (BME) (Trevigen); natural scaffolds comprising collagen (e.g., and collagen type IV), fibrin, bone sialoprotein, vitronectin (e.g., VITRONECTIN XF™ (STEMCELL Technologies), alginate, or laminin; synthetic polymeric scaffolds, e.g., comprising poly(2-(methacryloyloxy) ethyl dimethyl-(3-sulfopropyl)ammonium hydroxide) (PMEDSAH), polyacrylamide (PAM), poly(sodium 4-styrenesulfonate) (PSS), poly(methyl vinyletheralt-maleic anhydride), or poly(ethylene glycol) (PEG) hydrogels (e.g., photo-crosslinked or enzymatically crosslinked PEG-vinyl sulfone (PEG-VS), photopolymerizable PEG thiol-ene hydrogel scaffolds with cysteine-flanked MMP-sensitive crosslinks, or MMPdegradable, RGD-functionalized PEG hydrogel scaffolds factor-XIIa-mediated crosslinked peptide-functionalized PEG monomers), or combinations thereof. A number of suitable scaffolds are known in the art. See, e.g., Cruz-Acuña and García, Matrix Biol. 2017 January; 57-58( ):324-333; Murrow et al., Development. 2017; 144:998-1007; Murphy et al., Nat Mater. 2014; 13:547-557; Nguyen et al., Nat Biomed Eng. 2017; 1: 0096; and Aisenbrey and Murphy, Nature Reviews Materials 5:539-551 (2020), and references cited therein. In some embodiments, the hydrogel scaffold composition comprises one or more growth factors.

Media suitable for expansion of HSCs are known in the art; see, e.g., Chagraoui et al., PLOS One. 2019; 14(11): e0224900; Boitano et al., Science. 2010 Sep. 10; 329(5997): 1345-1348; Zhang and Lodish, Curr Opin Hematol. 2008 July; 15(4): 307-311. In some embodiments, the media for expansion of HSC includes stem cell factor (SCF), and optionally one or more of thrombopoietin (TPO), fms related receptor tyrosine kinase 3 (Flt3), Interleukin 6 (IL6), and an aryl hydrocarbon receptor (AHR) antagonist, and optionally UM729. In some embodiments, the media comprises the following:

| HSC Cocktail | Exemplary Concentration |
|---|---|
| Human thrombopoietin (TPO) | 10-250, e.g., 80-120, e.g., 100 ng/mL |
| Human Stem cell factor (SCF) | 10-250, e.g., 80-120, e.g., 100 ng/mL |
| Human fms related receptor | 10-250, e.g., 80-120, e.g., 100 ng/mL |
| tyrosine kinase 3 (Flt3) | |
| Human Interleukin 6 (IL6) | 10-250, e.g., 80-120, e.g., 100 ng/mL |
| StemRegenin 1 (SR1) | 10-250, e.g., 60-100, e.g., 0.75 uM |
| UM 729 | 5-50, e.g., 20-40, e.g., 35 nM |

A number of AHR antagonists are known in the art and can be used in the present methods, e.g., BAY-218 ((S)-6-(4-chlorophenyl)-2-(3-fluorophenyl)-N-(1-hydroxypropan-2-yl)-3-oxo-2,3-dihydropyridazine-4-carboxamide), PDM-11 ((E)-5-[2-(4-chlorophenyl)ethenyl]-1,3-dimethoxyphenyl), PDM2 (1,3-dichloro-5-[(1E)-2-(4-chlorophenyl)ethenyl]-benzene), GNF351 (N-(2-(3H-Indol-3-yl)ethyl)-9-isopropyl-2-(5-methyl-3-pyridyl)-7H-purin-6-amine), CH-223191 (2-Methyl-2H-pyrazole-3-carboxylic acid-(2-methyl-4-o-tolyl-azophenyl)-amide), and BAY 2416964; commercial sources for AHR antagonists include Selleck Chemicals, Calbiochem, and Sigma-Aldrich. See, e.g., Boitano et al., Science. 2010 Sep. 10; 329(5997):1345-8. In some embodiments, the AHR antagonist is StemRegenin 1 (SR1) (4-(2-(2-(Benzo[b]thiophen-3-yl)-9-isopropyl-9H-purin-6-ylamino)ethyl)phenol).

In some embodiments, the media comprises UM 729 (Methyl 4-{[3-(1-piperidinyl)propyl]amino}-1H-pyrimido[4,5-b]indole-7-carboxylate), a pyrimido-indole derivative that enhances HSC self-renewal in vitro. While not an inhibitor of the aryl hydrocarbon receptor (AHR) pathway, UM 729 synergizes with AHR antagonists.

Recombinant thrombopoietin (TPO), stem cell factor (SCF), fms related receptor tyrosine kinase 3 (Flt3), and Interleukin 6 (IL6) are all commercially available. Preferably, human thrombopoietin (TPO), human stem cell factor (SCF), human fms related receptor tyrosine kinase 3 (Flt3), and human Interleukin 6 (IL6) are used.

In some embodiments, approximately 1-2 million cells can be recovered, e.g., after 10-20, e.g., 12-18, or 14-17 days of culture, from a starting sample of about 60 pieces of tissue. Of that 1-2 million, about 1-2% are HSC based on magnet bead purification. Thus in some embodiments, the population comprises 10,000-20,000 HSCs. The presence of HSCs in this population can be validated, e.g., through flow cytometry or magnetic sorting methods, to identify and optionally isolate cell populations based on defined cell surface markers unique for hematopoietic stem cells (e.g., CD34+, CD45dim, CD90+, CD38−, and/or Lineage−). The potency of HSCs obtained using these methods has been validated through analysis of their ability to differentiate into myeloid and lymphoid cells in vitro as shown in Example 1.

Previously, it was not known that the human yellow bone marrow contained HSCs, as these cells are very rare, and the adipose content of the yellow marrow prevents them from being directly isolated by current techniques (Kricun et al., Skeletal Radiol. 1985; 14(1):10-9. As shown herein, placing yellow marrow fragments under specific 3-dimensional culture conditions, which include a hydrogel and pro-angiogenic growth factors and HSC expansion cytokines, results in proliferation of HSCs, proving that they exist in this compartment. The HSCs that proliferate from yellow marrow fragments can be recovered, display canonical HSC markers detected by FACS, and can differentiate into blood cell lineages in-vitro. Currently, the use of HSCs for cancer therapies requires human subjects to undergo multiple injections of G-CSFs and subsequent peripheral blood collection that is then dialyzed for HSC enrichment. The present methods eliminate the risk of multiple injections of G-CSFs, serial blood collection and time-intensive process of dialysis, as we can generate HSCs from subjects by removing a small fraction of the yellow marrow through a punch biopsy of their long bones, which can be completed in a single short visit.

The present methods can be used, e.g., to obtain sufficient amounts of patient-specific HSCs for autologous transplantation procedures, as well as for stocking in a tissue bank for heterologous transplantation. The HSCs can be differentiated to different cell types, including immune cells, e.g., using methods known in the art; see, e.g., Gallagher et al., Diabetes. 2014 Nov. 3. pii: DB_140872; Kittan et al., PLOS One. 2013 Oct. 21; 8(10):e78045; Elliot et al., 1990, J. Immunol. 145: 167; Kaufman et al., PNAS Sep. 11, 2001 98 (19) 10716-10721. For example, the HSCs can be cultured in media comprising methylcellulose in Iscove's MDM, Fetal bovine serum, Bovine serum albumin, 2-Mercaptoethanol, human stem cell factor (SCF), human interleukin 3 (IL-3), human erythropoietin (EPO), human granulocyte colony-stimulating factor (G-CSF), and human granulocyte-macrophage colony-stimulating factor (GM-CSF), to support differentiation to erythroid progenitor cells (BFU-E and CFU-E), granulocyte-macrophage progenitor cells (CFU-GM, CFU-G and CFU-M), and multipotential granulocyte, erythroid, macrophage, megakaryocyte progenitor cells (CFU-GEMM). The culture conditions can be altered to promote differentiation to a selected cell type, e.g., using methods known in the art. For example the HSCs can be cultured in the presence of human stem cell factor (SCF), human interleukin 3 (IL-3), and human erythropoietin (EPO) to generate erythroid progenitor cells. As another example, the HSCs can be cultured in the presence of human stem cell factor (SCF), human interleukin 3 (IL-3), and granulocyte-macrophage colony-stimulating factor (GM-CSF) to generate granulocyte-macrophage progenitor cells (CFU-GM, CFU-G and CFU-M). Methods to generate functional immune cells are known in the art, see, e.g., Payuhakrit et al., EXCLI J. 2015; 14: 1031-1039; Guo et al., Blood Science: 2(1):22-26 (January 2020).

The HSCs, or cells derived therefrom, can be genetically modified, and reintroduced for therapeutic purposes. In some embodiments, the HSCs can be differentiated to immune cells, and reintroduced to a subject in need thereof. For example, the HSCs can be differentiated to erythroid progenitor cells, granulocyte-macrophage progenitor cells, multipotential granulocyte, erythroid, macrophage, megakaryocyte progenitor cells, lymphoid progenitor cells (Common Lymphoid Progenitors (CLPs)), B cells, T cells, or NK cells. T Cells or NK cells, and other cell types, can be genetically modified (e.g., to express a chimeric antigen receptor), and introduced into a subject for cancer immunotherapy. The cells can be administered by any suitable route, e.g., by intravenous or intraarterial transfusion, intrathecally, or by infusion via catheter into a tissue. These cells can be administered, e.g., to a subject who is deficient from a variety of disease, infections, or blood loss. In some embodiments, the HSCs are optionally differentiated, e.g., to monocytes and/or anti-inflammatory macrophages (see, e.g., Boniakowski et al., J Immunol Jul. 1, 2017, 199 (1) 17-24) and applied to a wound, e.g., in a subject who has diabetes. In some embodiments, the cells are administered locally, e.g., by injection into or on the wound itself. In some embodiments, the cells are autologous to mitigate chances of immune rejection. If the cells are differentiated, they can be further purified if a pure population of cells is desired.

### Examples

The invention is further described in the following examples, which do not limit the scope of the invention described in the claims.

### Materials and Methods

The following materials and methods were used in Example 1, below.

### Exemplary Procedure for Human Bone Marrow and Fat Tissue Explant Tissue Harvesting and Embedding in Matrigel:

### Day Before Tissue Embedding: Reagent Prep

1. Thaw Matrigel (Corning, Ref #35623) in cold room (Stored in −20 C per manufacture's recommendation)
2. Aliquot ˜50 mL of media comprising pro-angiogenic factors (EGF, IGF, VEGF, and FGFb), stored in 4 C for tissue collection
3. HSC stock cocktail aliquot to make HSC media for after embedding
   a. Resuspend cytokines in 0.1% BSA in distilled water filter with 22 um filter
   b. Make HSC media fresh weekly for subsequent cell culture media change

| | | | | | | Aliquots of | |
|---|---|---|---|---|---|---|---|
| | | | | | | Stock Need | |
| HSC Stock | | | Stock | | | to make 20 mL | |
| Cocktail | Cat # | Original | Dilution | Final Conc | | of HSC media | |
| Recombinant | 300-18 | 10 ug | 2 ug/mL | 100 | ng/mL | 1 | mL |
| Human TPO | | | in 5 mL | | | | |
| Recombinant | 300-07 | 10 ug | 2 ug/mL | 100 | ng/mL | 1 | mL |
| Human SCF | | | in 5 mL | | | | |
| Recombinant | 300-19 | 10 ug | 2 ug/mL | 100 | ng/mL | 1 | mL |
| Human Flt3 | | | in 5 mL | | | | |
| Recombinant | 200-06 | 20 ug | 2 ug/mL | 100 | ng/mL | 1 | mL |
| Human IL6 | | | in 5 mL | | | | |
| StemRegenin | 1224999 | | 10 mM | 0.75 | uM | 1.2 | uL |
| 1 (SR1) | | | | | | | |
| UM 729 | StemCell | | 1 mM | 35 | nM | 0.8 | uL |

For the UM729: 250 ug resuspended in 551 ul DMSO->gives a 1 mM solution.
Use 0.2 ul in 5 ml to get 35 nM final concentration
For SR-1: 1 g resuspended in 230 ul DMSO->gives a 10 mM solution.
Use 0.3 ul in 5 ml to get a 0.75 uM final concentration.
   HSC Media=Add HSC Cytokine Stock Dilutional Aliquots at Concentration Above with M131 Complete Media to Achieve Desired Volume

### Harvesting Bone Marrow Tissue Explant Samples

1. Use ronjour to scoop out RED BONE MARROW (BMR) from distal tibia or femur-->place in specimen cup with media comprising pro-angiogenic factors.
2. Use curvette to scoop out YELLOW BONE MARROW (BMY) from shaft of tibia or femur-->place in specimen cup with Corvera complete media
0. Use scalpel to excise SQ fat (BMS)-->place in specimen cup with Corvera complete media.
1. Store on ice.
2. Collect red and yellow marrow and SQ to store for RNA and tissue snap freeze, OCT, and formaldehyde
   a. RNAse spray all materials
   b. Collect ˜100 mg red (BMR), yellow (BMY) bone marrow tissue and SQ fat (BMS) in RNAse treated foil squares to wrap tissue and snap freeze in liquid nitrogen
      i. Collected 4 samples of each depot

### Matrigel Embedding of Human Tissue Explants

1. Using #10 scalpel and forceps in tissue culture hood, mince tissue in media in 10 cm plate, want to 1 mm³ pieces
2. Select 180 ˜1 mm³ tissue pieces to imbed in a 10 cm plate with 7 mL Matrigel on an ice block in hood each for BMR and BMY
3. Select 30 ˜1 mm tissue pieces to imbed 1 well with 1.2 mL Matrigel (Corning) in 6-well plate on ice plate in hood—total 90 explants of BMS in a total of 3 wells
4. Incubate in 37 C for 30 minutes to solidify Matrigel
5. Add 2 mL of HSC medial to each well in 6-well and 10 mL of media for 10 cm plates
6. Full media change the next day
7. Change ½ media every other day until 14-17 days in culture
   Exemplary Procedure for Bone Marrow Tissue Explant Cell Digestion from Matrigel Culture: Cells Harvested 14-16 Days in Matrigel Culture

### Day Before—Material Preparation:

Thaw Dispase (Corning, Ref #354235) in cold room or 4 C
Will need volume of dispase equal to initial volume of Matrigel used for embedding tissue explants
Dispase stored in −20 C per manufacture's instructions

### Day of Procedure:

1. Collect 1 mL of media from culture and store in −80° C. for potential protein analysis in future
2. Wash cell culture with PBS×2
   1. 2 mL per well in a 6-well plate or 10 mL per 10 cm plate
3. Add equal amount of Dispase to Matrigel (1.2 mL for wells in 6-well plate and 7 mL for 10 cm plates)
4. Incubate in 37 C for ˜40 min total and agitate cells/Matrigel during incubation by pipetting up and down with 1000 uL cut end pipette tip at 20 min and 40 min time points.
5. Added 240 uL of Trypsin (10×) with 20 mg/mL of Collagenase (sterilized through a 0.22 um syringe filter) to 6-well and 1.4 mL to 10 cm plates for 14 min total (Final conc. 1× Trypsin with 2 mg/mL of Collagenase).
6. Agitate cells by pipetting up and down with 1000 uL cut end pipette tip at 7 min and 14 min time points
7. Transfer the contents through a 100 um cell strainer into a 50 mL conical tube.
8. Add 10 mL M131 wash media to 10 cm plates or 2 mL to 6-well plate to wash/collect cells stuck to the surface, and again transfer through the 100 um strainer to wash off the explants into the same 50 mL conical tube to stop the digestion.
9. Add additional media to through filter to get final volume to 30-20 mL and wash off remaining cells on explant
10. Centrifuge at 21 C at 800 rcf for 30 min
11. Aspirate media.
12. Wash pellets with 5 mL of media for BMR and BMY and for BMS
13. Manual cell count on hemocytometer under microscope at 10× objective
14. Centrifuge at 21 C at 800 ref for 10 min
15. Aspirate cell culture media and resuspend in 5 mL for BMR, BMY, and BMS for subsequent experiments using single cell suspension

### Exemplary Procedure for FACS Analysis of Bone Marrow Tissue Explant Cell:

1. Wash single cell suspension collected from SOP for BONE MARROW TISSUE EXPLANT CELL DIGESTION FROM MATRIGEL CULTURE with 1 mL of FACS buffer (FACS Buffer: 500 ml 1×pbs+5 ml pen/strep+5 ml FBS)
2. Spin 300 g, 5 minutes, 4° C.
3. Aspirate out supernatant
4. Gently wash cells by resuspending in 1 ml of FACS buffer
5. Spin 300 g, 5 minutes, 4° C.
6. Repeat Wash 2 more times
7. Prepare antibody Mastermix (1:50 Ab dilution, BioLegend)

| | uL per | | |
|---|---|---|---|
| | sample | #samples | MM |
| CD34 PE (Cat#343605) | 1 | | |
| CD38 PE-Cy7 (Cat#356607) | 1 | | |
| LINEAGE APC (CD3, 14, 16, 19, | 1 | | |
| 20, 56) (Fisher Cat#558074) | | | |
| CD45-AF700 (Cat#304023) | 1 | | |
| CD90 FITC (Cat#328107) | 1 | | |
| FACS BUFFER | 35 | | |
| TOTAL | 40 | | |

8. Resuspend each sample cell pellet in 50 uL of FACS Buffer
9. Combine 5 uL of each cell sample to get POOLED CELLS
10. Add 45 uL of FACS Buffer to POOLED CELLS
11. EXPERIMENT SAMPLE: Add 40 ul Master Mix to each sample and incubate on ice (covered) for 20 minutes
12. Prepare UltraComp eBeads compensation beads for fluorescence standard (Invitrogen, Catalog #01-2222-42)
   Aliquot 2 drops of beads into a 1.5 mL eppie tube
   Wash with 1 mL of PBS
   Spin 300 g, 5 minutes, 4° C.
   Aspirate media and resuspend in 350 uL PBS
   Aliquot 50 uL into fresh 1.5 mL eppie (label #7-11)
13. BEADS SINGLE STAIN CONTROL: Add 1 ul of antibody to each respective tubes #5-11 and incubate on ice (covered) for 20 minutes
14. Add 1 mL FACS buffer to all tubes to wash
15. Spin 300 g, 5 minutes, 4° C.
16. Aspirate supernatant from tubes
17. Resuspend UNSTAINED SAMPLES (#1 AND #5) in 200 uL of FACS buffer
18. Add 0.5 uL of FVD Viability stain (Invitrogen Cat #136430192)+200 uL of FACS buffer indicated tubes below

19. Incubate for 30 minutes in dark on ice
20. Spin 300 g, 5 minutes, 4° C.
21. Aspirate and resuspend each sample in 200 uL of PBS
22. Resuspend each sample in 200 uL of FACS buffer add to all tubes then add 200 uL of 4% PFA to get final concentration 2%
   For 5 ml of 4% PFA: 1000 ul 16% Paraformaldehyde
      4 ml FACS buffer
23. Incubate on ice for 10 minutes in dark
24. Add 1 mL of FACS buffer to all tubes and spin 500 g, 5 minutes, 4° C.
25. Resuspend pellet in 50 uL of HBSS
26. Transfer to FACS tubes and cover tubes with parafilm then wrap rack in foil and store at 4° C.--->transport to FACS core

### Exemplary Procedure for CD34 Cell Isolation of Bone Marrow Tissue Explant Cell: Modified EasySep Human CD34 Selection Kit II (StemCell Cat #17856)

1. Wash single cell suspension collected from SOP for BONE MARROW TISSUE EXPLANT CELL DIGESTION FROM MATRIGEL CULTURE with 1 mL of recommended media (500 mL PBS+2% FBS (20 mL)+1 mM EDTA (1 mL))
2. Resuspend cells in 100 uL of Recommended Media
   Cells typically <10\hat{ }6 so resuspend pellet in 100 ul of recommended medium
3. Add 10 uL Selection Cocktail to each sample
4. Mix and incubate at room temperature for 10 minutes
5. Vortex RapidSpheres for 30 seconds
6. Add 75 uL of RapidSpheres to sample (75 ul to each sample)
7. Mix and incubate at room temperature for 5 minutes
8. Add Recommended Medium to bring up to 2.5 mL volume in a polystyrene 5 mL tube
9. Mix by gently pipetting up and down 2-3 times
10. Washing Step: Place tube in EasySep magnet (without top) and incubate for 3 minutes at room temperature
11. Collect flow through supernatant (while tube is still in the magnet—leave inverted for 2-3 seconds, do not shake or blot off), what is left in the tube are the isolated cells
12. Repeat above washing steps for a total of 3×:
13. Resuspend cells in 500-200 uL of recommended media depending on anticipated cells recover (typically ˜1% of total cells input).
14. Count cells with hemocytometer under 10× objective

### Exemplary Procedure for Colony Forming Unit Assay to Test Pluripotent Potency of CD34 Cell Isolation of Bone Marrow Tissue Explant Cell: MethoCult H4034 Optimum, Cat #04044

1. Use CD34+ single cell suspension collected from SOP for CD34 CELL ISOLATION OF BONE MARROW TISSUE EXPLANT CELL
2. Thaw 3 vial of 3 mL MethoCult H4034 Optimum @4 C fridge (can be done the night before) or at room temperature
3. Using a blunt end 16 G needle and 3 ml syringe, aliquot 1.5 mL of MethoCult Optimum into 2 mL tubes in tissue culture hood
4. Add 20-50K of each cell sample to 1.5 mL aliquots of MethoCult Optimum
5. Vortex tube and let stand for ˜5 minutes to allow bubbles to rise to top
6. Use 16 G needle and syringe to transfer MethoCult Medium/Cell mixture to 6-well SmartDish (StemCell Cat #27370)
7. Seal the 6-well SmartDish with parafilm
8. Incubate with surrounding 35 mm sterile water in a Pyrex dish pan covered with aluminum foil to maintain humidity for 10-14 days
9. Monitor for colony growth on Day 10-14 and count number of erythroid progenitors (CFU-E), granulocyte-macrophage progenitors (CFU-GEMM) using STEMgrid-6 (StemCell Cat #27000).
   Exemplary Procedure for Bone Marrow Tissue Explant Cell RNA Extraction, cDNA Synthesis, and Quantitative PCR for LEPR

### RNA Extraction:

### Day 1

### Samples Frozen in 1 mL Trizol Using TissueLyser (Bead Beater)

1. Resuspend ˜500,000 tissue explant cells in 1 mL of Trizol
2. Dissociate the cells in QIAGEN tissuelyser for 3 min at 30 Hz
3. Transfer the all the lysate into the phase lock gel-heavy tube with 1.0 uL glycogen or glycoblue.
4. Add 200 uL ice cold chloroform, invert the samples for 15 sec, incubate for 5 min @RT, and spin the tube for 15 min @12,000 g @4° C.
5. Carefully transfer the clear aqueous phase to a new tube
6. Add equal volume of 100% isopropanol (500 mL) to the tube, invert to mix
7. Precipitate the RNA overnight @−20° C.

### Day 2

9. Spin the tube for 30 min @12,000 g @4° C.
10. Carefully remove the supernatant, AND observe a small blue pellet
11. Remove the remaining supernatant with a 200 uL pipette
12. Wash the pellet with 500 uL 80% EtOH (make sure the pellet is dislodged from the side of the tube, add 80% EtOH quickly, DO NOT VORTEX)
13. Spin the tube for 10 min @4° C. @7500 g
14. Remove the EtOH by repeating steps 2-3
15. Air dry the pellet @RT by lying the tube on its side for 30 min
16. Resuspend the pellet in 20 uL RNase-free water and measure RNA concentration on Nanodrop
17. Store in −80 C
   cDNA Synthesis:
   Make cDNA-Invitrogen Superscript III qRT-PCR
Use 1 ug of RNA per reaction
1. Combine the following

| | | |
|---|---|---|
| 2x RT reaction Mix | 10 | ul |
| RT Enzyme Mix. | 2 | uL |
| RNA | 1 | ug |

| | |
|---|---|
| DEPC-water | to 20 ul |

2. Gently mix tube and incubate at 25 C for 10 min
3. Incubate at 50 C for 30 min
4. Terminate rxn at 85 C at 5 min and chill on ice-->placed in-20 fridge o/n
5. Add 1 uL (2 U) of E. Coli Rnase H and incubate at 37 C for 20 min

*cDNA is made from 1 ug of RNA and diluted 1:4 (20 ul cDNA rxn+60 ul water)

qPCR for LEPR:

PRIMERS

| Primer | Sequence | SEQ ID NO: |
|---|---|---|
| LEPR Fwd | GCTATTTTGGGAAGATGT | 1 |
| LEPR Rev | TGCCTGGGCCTCTATCTC | 2 |
| RPL4 Fwd | GCCTGCTGTATTCAAGGCTC | 3 |
| RPL4 Rev | GGTTGGTGCAAACATTCGGC | 4 |

### Make Master Mix for Each Primer Sets:

| Rxn# | FWD Primer | REV Primer |
|---|---|---|
| 1 | LEPR Fwd | LEPR Rev |
| 2 | RPL4 Fwd | RPL4 Rev |

| | Final | 1x | | #25 Rxn for Master | |
|---|---|---|---|---|---|
| Components | Concentration | Volume | | for each Primer Set | |
| H2O | — | 7.2 | ul | 187.2 | uL |
| KAPA SYBR | 1x | 10 | ul | 260 | uL |
| Fast MM (2x) | | | | | |
| 10 uM Primer F | 200 nM | 0.4 | ul | 10.4 | uL |
| 10 uM Primer R | 200 nM | 0.4 | ul | 10.4 | uL |

| | | | | |
|---|---|---|---|---|
| Template cDNA | cDNA dilution | 2.0 | ul | aliquot each |
| | from 1 ug of RNA | | | |
| Total | | 20 | ul | *aliquot 18 uL of |
| | | | | MM to each well |

*cDNA is made from 1 ug of RNA and diluted 1:4 (20 ul cDNA rxn + 60 ul water)

### PCR Cycle Time:

| Steps | Temp | Duration | | Cycles |
|---|---|---|---|---|
| 1: Enzyme activation | 50° C. | 2 | min | |
| 2: Enzyme activation | 95° C. | 3 | min | |
| 3: Denaturation | 95° C. | 15 | sec | 40x Step 3-5 |
| 4: Annealing | 60° C. | 15 | sec | |
| 5: Extension | 72° C. | 30 | sec | |
| 6: Dissociation | 72° C. | 10 | in | |

| | | |
|---|---|---|
| 7: Melting curve | | |
| 8: Completion | 4° C. | Forever |

Generation of 3D tissue explant co-culture system: Methods for the harvesting and the culture adipose tissue explants were previously published (Min et al., Nat Med. 2016 March; 22 (3): 312-318). Human tissues were donated from consented adult patients undergoing lower extremity amputation surgery at University of Massachusetts Medical Center and were subjected to harvesting within one to two hours. Explants of approximately 1 cm³ in size were embedded in Matrigel Matrix (Cat #356231, Corning) per 10 cm dish with EGM-2 MV (Cat #CC-3156,CC-4147, Lonza) media supplementation. The excised tissue were embedded in Matrigel (180 explants/10 cm dish) or (30 explants/6-well dish) and cultured for 14-17 days as described before. Addition of 100 ng/ml stem cell factor (SCF), 100 ng/ml interleukin-6 (IL-6), 100 ng/mL thyroperoxidase (TPO), 100 ng/mL Fms-like tyrosine kinase 3 ligand (Flt3), 35 mM pyrimido-indole derivative (UM171 or UM729), and 0.75 uM StemRegenin (SR-1) was added to culture to promote HSC expansion. After 12-17 days in Matrigel, the progenitors in explants were recovered using Dispase (Cat #354235, Corning) for one hour followed by additional 14 minutes of Trypsin-EDTA (Cat #15400-054, Gibco) and Collagenase I (Cat #LS004197, Worthington).

CD34 Positive Cell Enrichment: CD34⁺ cells were enriched from de novo progenitor cells collected from each respective 3D hydrogel tissue explant culture using the EasySep Human CD34 Selection (StemCell Technologies. #17856). HUCB CD34⁺ cells were isolated from whole blood using Lymphoprep (StemCell Technologies, #07801) to collect mononuclear cells then purified using EasySep Human Cord Blood CD34 Selection (StemCell Technologies, #17896). HUCB CD34⁺ cells were then seeded in Matrigel Matrix (Cat #356231, Corning) for 14-17 days for HSC expansion. After HSC culture expansion, HUCB CD34⁺ cells in Matrigel Matrix were then harvested using Dispase (Cat #354235, Corning) for 40 minutes followed by additional 14 minutes of Trypsin-EDTA (Cat #15400-054, Gibco) and Collagenase I (Cat #LS004197, Worthington). HUCB CD34⁺ cells harvested from Matrigel Matrix were subsequently enriched using EasySep Human Cord Blood CD34 Selection (StemCell Technologies, #17896). Enriched CD34⁺ were then either used for FACS, colony forming unit assay, or in vitro differentiation.

Flow Cytometry: Flow cytometry assays were performed on BD LSR. Viable cells were determined with live/dead cell marker (Invitrogen™ eBioscience™ Fixable Viability Dye eFluor™ 455UV. ThermoFisher Scientific #65-0868-14). The following fluorescent conjugated antibodies at a 1:50 dilution were used:

| BioLegend Cat# | |
|---|---|
| 348803 | Human lineage APC |
| 304023 | Alexa Fluor 700 ™ anti-human CD45 Antibody |
| 343605 | PE anti-human CD34 |
| 356607 | PE/Cy7 anti-human CD38 |
| 328107 | FITC anti-human CD90 (Thy1) |
| 305013 | APC anti-human CD64 |
| 333611 | Brilliant Violet 421 anti-human CD163 |
| 367115 | FITC anti-human CD14 |
| 343503 | FITC anti-human CD34 |
| 336107 | PE anti-human CD93 |

Flow cytometry data analysis was performed with FACS Diva 6.1 software and analysis done on FloJo software (10.8.1).

HSC-induced Differentiation Towards Monocytes and Macrophages: CD34 positive cells enriched using magnet beads (StemCell) were cultured in complete RPMI 1640 medium (10% FBS, 100 U/ml penicillin, 100 U/ml streptomycin, with 50 ng/ml SCF, 20 ng/ml TPO, and 50 ng/ml Flt3 with 25 ng/ml M-CSF (Peprotech). After 5-7 days, cells were induced to differentiate towards M1 macrophage by changing the medium to the M1 induction medium (complete RPMI 1640 medium, 5% FBS, 100 U/ml penicillin, 100 U/ml streptomycin, and 5 ng/mL IFN-gamma for 24-36 hours. Cells were then collected and stained FACS analysis. Stained cells were run through a BD FACSCalibur flow cytometer and data were analyzed using the FlowJo software. All the cytokines were purchased from Peprotech and all the antibodies were purchased from BD Biosciences.

### Example 1. Human Bone Marrow Tissue Explant Collection and In Vitro Cell Expansion

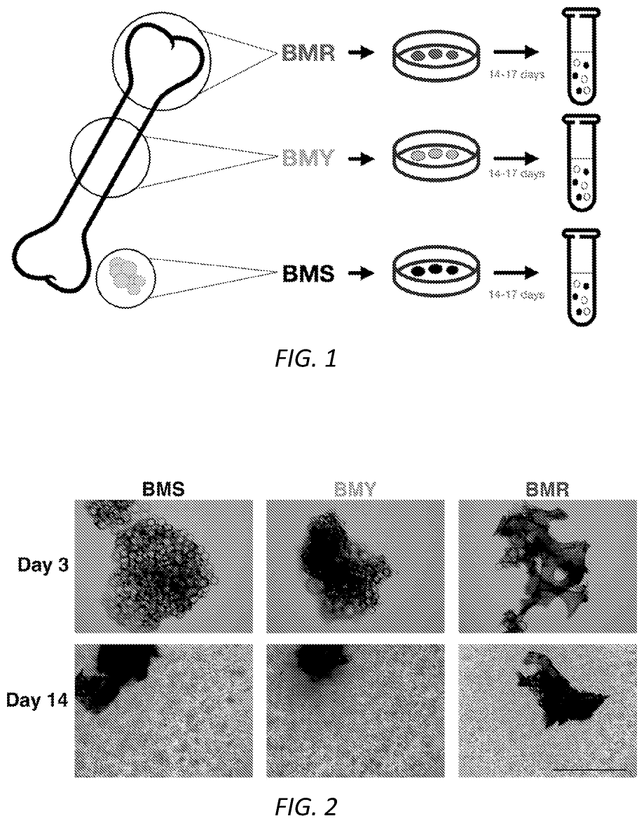

As shown in FIG. 1, discarded human bone marrow and lower extremity subcutaneous fat tissue were collected from patients undergoing lower extremity amputation. Two different anatomical regions of bone marrow tissue explants were collected, bone marrow red (BMR) and bone marrow yellow (BMY). Lower limb subcutaneous fat tissue explant (BMS) was also collected. Tissue explants were cut into uniform size and embedded in Matrigel (Corning, Ref 356231) and cultured in pro-angiogenic media (e.g., comprising EGF, IGF, VEGF, and FGFb), with a cytokine cocktail to promote hematopoietic stem cell (HSC) expansion. The media comprised:

| HSC Cocktail | Cat # | Final Conc | |
|---|---|---|---|
| Recombinant Human TPO | 300-18 | 100 | ng/mL |
| Recombinant Human SCF | 300-07 | 100 | ng/mL |
| Recombinant Human Flt3 | 300-19 | 100 | ng/mL |
| Recombinant Human IL6 | 200-06 | 100 | ng/mL |
| StemRegenin 1 (SR1) | 1224999 | 0.75 | uM |
| UM 729 | StemCell | 35 | nM |

This human bone marrow three-dimensional culture system provides a unique method for 1) bone marrow cell expansion (up to 2 million cells from 60 pieces of 1 mm³ tissue explants) and 2) HSC culture in a system that will maintain the bone marrow niche micro-environment.

After 14-17 days in culture, the expanded tissue explant and cells were extracted from Matrigel using a digestion process to generate a single cell suspension for further analysis.

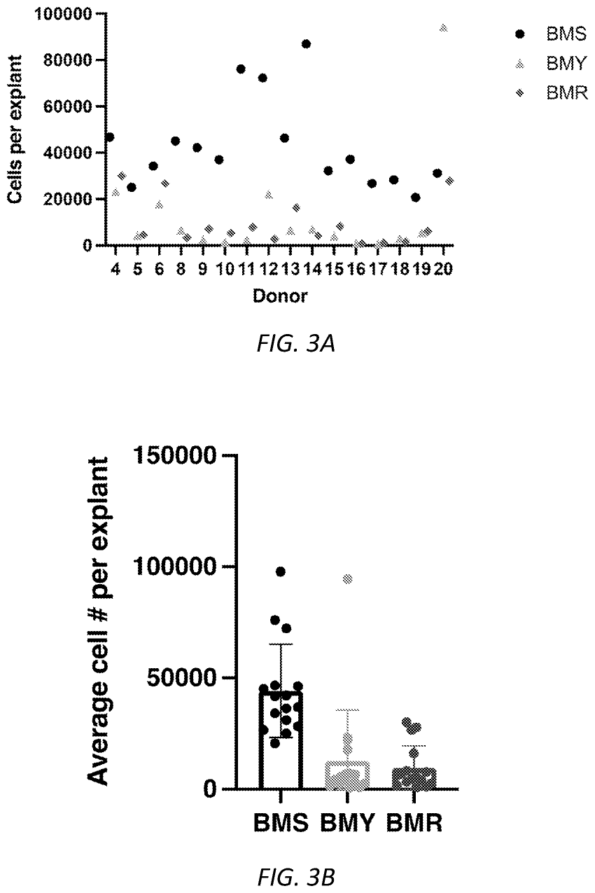

Manual quantification of the numbers of cells recovered after the Matrigel digestion process was done using a hemocytometer and microscopy imaging at 10× objective. The distribution of numbers of cells recovered from the three-dimensional culture system after 14-17 days in culture for lower limb subcutaneous fat tissue explant (BMS), bone marrow red (BMR), and bone marrow yellow (BMY) from respective donors is shown in FIG. 3A. The average number of tissue explant expanded cells collected after 14-17 days for growth is shown in FIG. 3B.

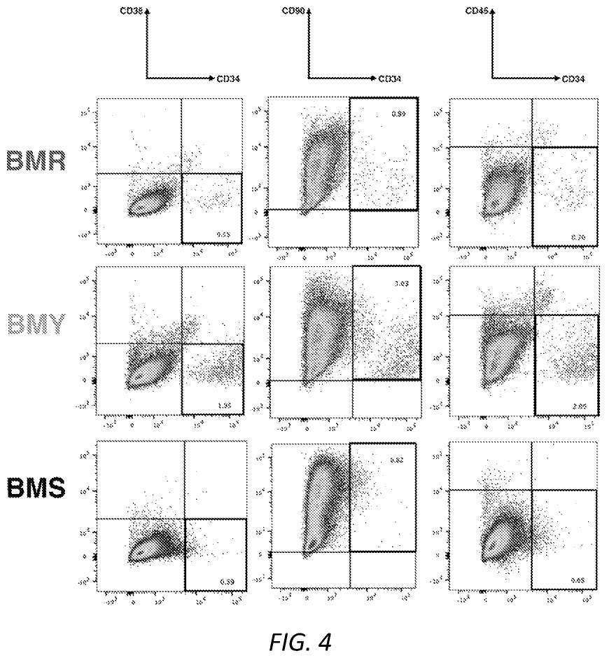

Flow cytometry experiments for identification and quantification of hematopoietic stem cells in human bone marrow tissue explant cell culture. After tissue explant cell expansion, the cells were extracted from the Matrigel using a digestion process to generate a single cell suspension. Single cell suspensions from BMR, BMY, and BMS tissue explant cell cultures were incubated with fluorescent antibodies that bind to common cell surface receptor markers used to identify hematopoietic stem cells (HSC defined as CD34+, CD38−, CD90+, and low levels of CD45+). The results are shown in FIG. 4.

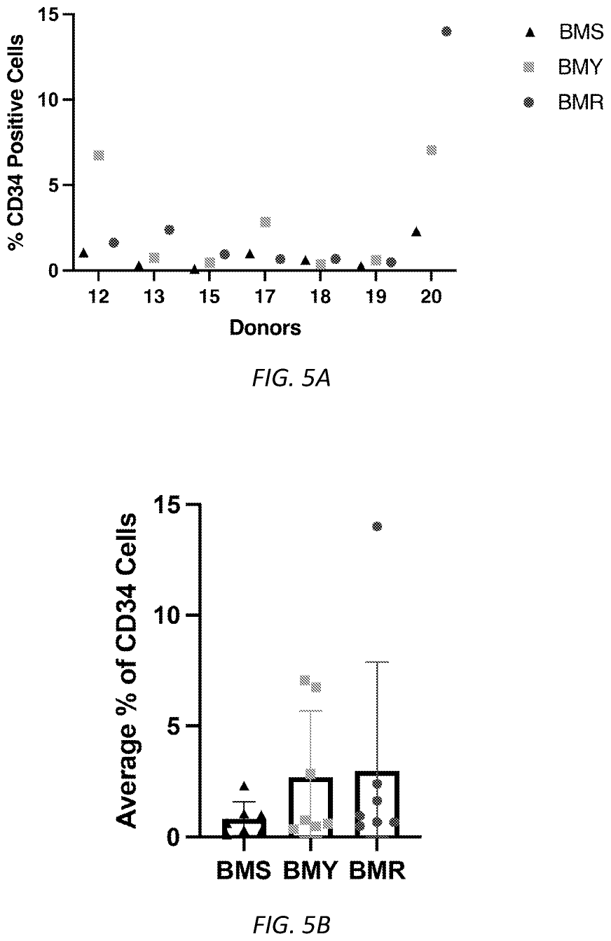

Isolation of hematopoietic stem cells in the human bone marrow tissue explant cell cultures was also evaluated. After tissue explant cell expansion, the cells were extracted from Matrigel using a digestion process to generate a single cell suspension. Single cell suspensions from BMS, BMY, and BMR tissue explant cell cultures were incubated with CD34 magnetic beads (StemCell, Kit #17856). Cells harboring CD34 receptors (conical marker for hematopoietic stem cells) will bind to the CD34 antibody coated magnetic beads and subsequently bind to the magnet test tube receptacle. This allows for isolation of CD34+ cells that are used for further experiments. FIG. 5A shows the percentage of CD34+ cells obtained from each respective donor, and 5B shows the average percent of CD34+ cells collected overall, from the three-dimensional culture system after 14-17 days in culture for lower limb subcutaneous fat tissue explant (BMS), bone marrow red (BMR), and bone marrow yellow (BMY) after 14-17 days for growth.

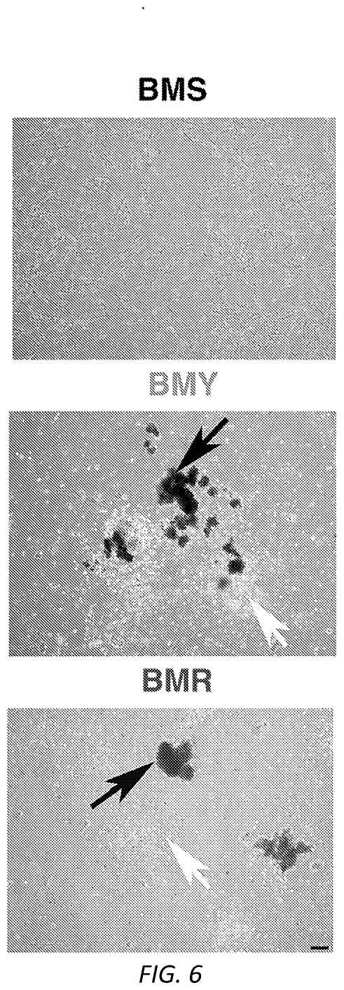

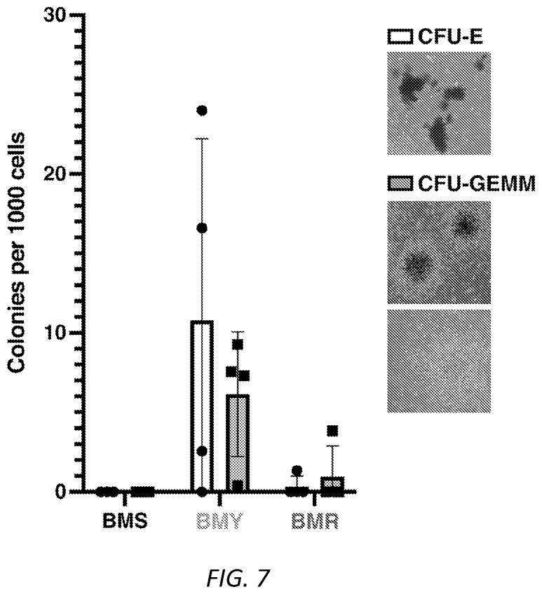

To determine whether erythroid and myeloid colonies could be formed from CD34+ cells isolated from human bone marrow tissue explant culture, subcutaneous fat (BMS), yellow bone marrow (BMY), and red bone marrow (BMR) tissue explants cells harboring CD34 receptors (conical marker for hematopoietic stem cells, HSC) were isolated using magnet beads (StemCell, Cat #17856). To verify and test for pluripotency function for HSC, CD34+ isolated cells were cultured in a colony forming unit assay (StemCell, MethoCult H4034 Optimum, Cat #04044) to test for a single origin cell to differentiate into both erythroid and myeloid blood cells. The results, shown in FIG. 6, showed that erythroid and myeloid colonies formed from CD34+ cells from both BMY and BMR.

Experiments were performed to quantify development of erythroid and granulocyte-myeloid-macrophage colonies from CD34+ Cells isolated from human bone marrow tissue explant cultures. Subcutaneous fat (BMS), yellow bone marrow (BMY), and red bone marrow (BMR) tissue explants cells harboring CD34 receptors (conical marker for hematopoietic stem cells, HSC) were isolated using magnetic beads (StemCell, Cat #17856). To test for pluripotency function for HSC, CD34+ isolated cells were cultured in a colony forming unit assay (StemCell, MethoCult H4034 Optimum, Cat #04044) to determine whether a single origin cell can differentiate into both erythroid and myeloid blood cells. The results, shown in FIG. 6, demonstrated development of erythroid and granulocyte-myeloid-macrophage colonies from CD34+ Cells isolated from yellow bone marrow.

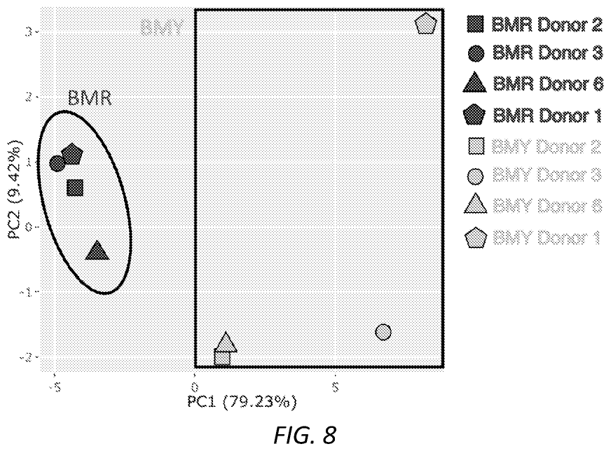

Principal component analysis (PCA) was performed to evaluate differential gene expression between red and yellow bone marrow. Bulk RNA-Seq was performed on cells expanded from yellow bone marrow (BMY) and red bone marrow (BMR) tissue explants after 14-17 days in culture from 4 patient donors. An average of 13 million reads per sample was referenced to the human genome to identify an average of over 20,000 genes per sample. Differential gene expression analysis was performed on the number of gene count data per sample using DeBrowser (UMMS Biocore). Significant differential gene expression was determined based on a padj cutoff of 0.01 or log fold change 2. The results are shown in FIG. 8.

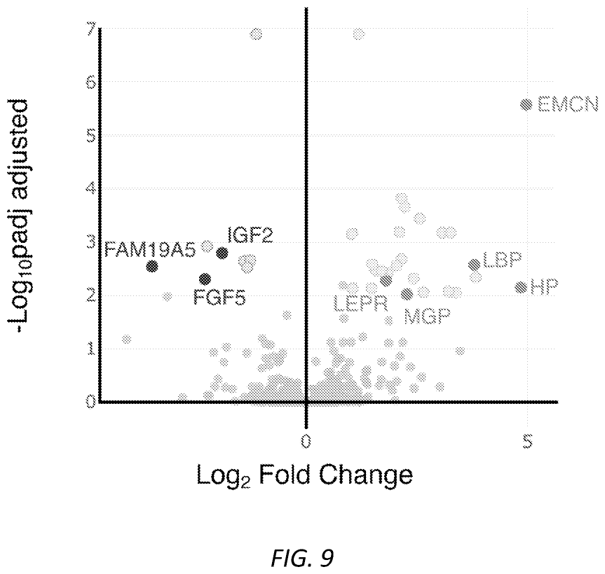

Genes that were differentially expressed between red and yellow bone marrow were evaluated. Bulk RNA-Seq was performed on cells expanded from yellow bone marrow (BMY) and red bone marrow (BMR) tissue explants after 14-17 days in culture from 4 patient donors. Differential gene expression analysis was performed on the number of gene count data per sample using DeBrowser (UMMS Biocore). Twenty-six differentially expressed genes were identified based on a padj cutoff of 0.01 or log fold change 2. The results, shown in FIG. 9, indicated substantially differential expression of genes including FAM19A5, IGF2, and FGF5 (which were higher in BMR-derived cells), as well as LEPR, MGP, LBP, HP, and EMCN (which were higher in MBY-derived cells).

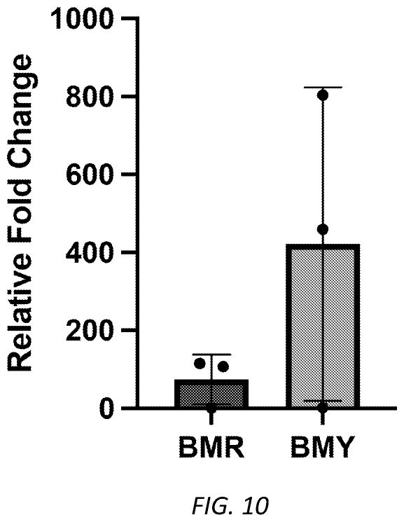

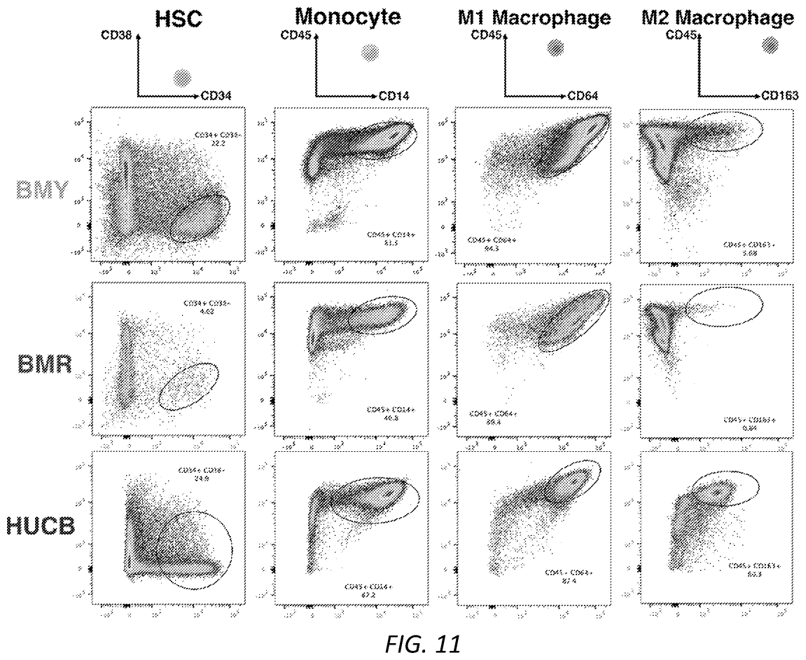

Leptin receptor gene expression was determined in cells generated from red and yellow bone marrow. The leptin receptor (Ob-Rb) gene was amplified from cDNA generated from red and yellow bone marrow tissue explant cell expansion cultured in HSC media. Expression fold change was normalized to housekeeping gene RPL4 and relative to the lowest expressing sample. The results, shown in FIG. 10, showed that leptin receptor expression was increased in cells from yellow marrow relative to cells from red marrow.

Cells obtained from yellow bone marrow (BMY), red bone marrow (BMR) tissue explants, or from human umbilical cord blood (HUCB), were differentiated into monocytes and macrophages. FACS analysis, shown in FIG. 11, demonstrated that these cells can functionally be differentiated into three different immune cell types (monocytes, M1 macrophage, and M2 macrophage) in a similar manner as human umbilical cord blood HSCs (HUCB).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of obtaining a population of hematopoietic stem cells (HSCs), the method comprising:
providing a sample comprising yellow bone marrow from a subject;
dividing the sample into pieces each about 0.5-2 mm³, preferably about 1 mm³;
embedding the pieces in a hydrogel with media for expansion of HSCs;
maintaining the pieces in culture for a time sufficient for expansion of the HSCs, and isolating the HSCs from the hydrogel,
thereby obtaining a population of HSCs.

2. The method of claim 1, wherein the sample is from a long bone of the subject.

3. The method of claim 1, wherein the subject is a mammal, e.g., a human.

4. The method of claim 1, wherein the hydrogel comprises MATRIGEL.

5. The method of claim 1, wherein the media for expansion of HSCs comprises:
(i) pro-angiogenic factors, preferably EGF, IGF, VEGF, and FGFb;
(ii) stem cell promoting factors, preferably stem cell factor (SCF), and optionally
(iii) one or more of thrombopoietin (TPO), fms related receptor tyrosine kinase 3 (Flt3), Interleukin 6 (IL6), an aryl hydrocarbon receptor (AHR) antagonist; and/or UM729.

6. The method of claim 5, wherein the AHR antagonist is StemRegenin 1 (SR1).

7. The method of claim 1, wherein the pieces are maintained in culture for at least 10, 12, or 14 days, or up to 17, 18, 20, or 24 days.

8. The method of claim 1, further comprising isolating cells that are CD34+, CD45dim, CD90+, CD38−, and/or Lineage−, optionally using cell sorting or magnetic beads.

9. The method of claim 1, wherein the population comprises 10,000-20,000 HSCs.

10. The method of claim 1, further comprising culturing the population of HSCs under conditions sufficient for differentiation into erythroid progenitor cells, granulocyte-macrophage progenitor cells, multipotential granulocyte, erythroid, macrophage, megakaryocyte progenitor cells, and/or lymphoid progenitor cells (Common Lymphoid Progenitors (CLPs)), or to B cells, T cells, or NK cells.

11. A method of treating a subject, the method comprising:
obtaining a population of HSCs by the method of claim 1;
differentiating the HSCs into a selected erythroid, lymphoid, or myeloid cell type;
and administering the differentiated cells to the subject.

12. The method of claim 11, wherein the yellow bone marrow is obtained from the subject to be treated.

13. The method of claim 11, wherein the subject is diabetic and has a wound, and the cells are differentiated.

14. The method of claim 13, wherein the cells are differentiated to immune cells, preferably to monocytes and/or macrophages, and administered to the wound.

15. A population of HSCs obtained by the method of claim 1.

16. (canceled)

17. A population of cells comprising erythroid progenitor cells, granulocyte-macrophage progenitor cells, multipotential granulocyte, erythroid, macrophage, megakaryocyte progenitor cells, lymphoid progenitor cells (Common Lymphoid Progenitors (CLPs)), B cells, T cells, or NK cells, obtained by the method of claim 10.

18. (canceled)
